(19) United States
(12) Patent Application Publication
(10) Pub. No.: US 2025/0327778 A1
(43) Pub. Date: Oct. 23, 2025

(54) FLUIDIC CONTROL SYSTEM

(71) Applicant: UNIVERSITY OF SOUTH CAROLINA, COLUMBIA, SC (US)

(72) Inventors: ZECHARIAH B. KITZHABER, COLUMBIA, SC (US); MICHAEL L. MYRICK, COLUMBIA, SC (US)

(21) Appl. No.: 19/184,451

(22) Filed: Apr. 21, 2025

Related U.S. Application Data
(60) Provisional application No. 63/636,388, filed on Apr. 19, 2024.

(51) Int. Cl. G01N 30/32; G01N 30/60; G01N 33/50

(52) U.S. Cl. CPC G01N 30/32; G01N 30/606; G01N 30/6078; G01N 33/5005; G01N 2030/324

(57) ABSTRACT

Described herein is an apparatus and method for operating said apparatus which may allow for the repeat analysis of a fluid sample. Said apparatus may comprise an inlet segment, an analysis segment, an outlet segment, a bypass segment, a holding segment and fluidic switches, wherein fluidic communication may be established between the holding inlet segment, analysis segment, outlet segment, bypass segment and holding segment.

### CROSS REFERENCE TO RELATED APPLICATION

This application claims filing benefit of U.S. Provisional Patent Application Ser. No. 63/636,388 having a filing date of Apr. 19, 2024, which is incorporated herein by reference for all purposes.

### BACKGROUND

Flow-through analyzers like flow cytometers are useful for analyzing samples like cell cultures, since they allow cells to be analyzed one-by-one while maintaining much higher throughput than a manual microscopic analysis. This makes them a common choice for studying phytoplankton, in particular for monitoring changes within a population. A major drawback of flow-through analyzers is that they typically require high concentrations (>1000 cell/mL). Additionally, by design, each measurement is a single-shot and there is no way to track individual cells after they have passed through the analyzer, so it is impossible to perform repeat analysis. This makes it difficult to distinguish between intercellular variability and intracellular variability when comparing a culture to itself at different points in time.

Sheath flow cytometers may be used to more closely analyze individual cells in a sample. In a sheath flow cytometer, a fluid containing cells is passed through a nozzle. Said nozzle is surrounded by sheath liquid injectors. The sheath liquid forms a laminar “sheath” around the fluid containing the cells. A downside of this approach is that low sample throughput is required in order to not disrupt the laminar flow of the sheath liquid.

### SUMMARY

According to some embodiments of the present disclosure an apparatus and method of operating said apparatus are described herein. In some embodiments of the present disclosure, the apparatus comprises an inlet segment comprising a first end, an analysis segment comprising a first end and a second end, an outlet segment comprising a first end, a bypass segment comprising a first end and a second end, a holding segment comprising a first end and a second end, and a first fluidic switch and a second fluidic switches. The first fluidic switch establishes fluidic communication from the first end of the inlet segment to the first end of the analysis segment, and from the first end of the holding segment to the first end of the bypass segment. Alternatively, the first fluidic switch may establish fluid communication from the first end of the inlet segment to the first end of the bypass segment, and from the first end of the holding segment to the first end of the analysis segment. The second fluidic switch establishes fluidic communication from the second end of the analysis segment to the first end of the outlet segment, and from the second end of the bypass segment to the second end of the holding segment. Alternatively, the second fluidic switch may establish fluid communication from the second end of the analysis segment to the second end of the holding segment, and from the second end of the bypass segment to the first end of the outlet segment.

### BRIEF DESCRIPTION OF THE FIGURES

A full and enabling disclosure of the present subject matter, including the best mode thereof to one of ordinary skill in the art, is set forth more particularly in the remainder of the specification, including reference to the accompanying figures in which:

FIG. 1 is a diagram of the repeat analysis apparatus described herein.

FIG. 2 is a diagram of the repeat analysis apparatus described herein in embodiments where the apparatus comprises an optional analysis instrument.

FIG. 3 is a diagram of the repeat analysis apparatus described herein in embodiments where the apparatus comprises an optional perturbation instrument in the holding segment.

FIG. 4 is a diagram of the repeat analysis apparatus described herein in embodiments where the apparatus comprises both an optional analysis instrument in the analysis segment and an optional perturbation instrument in the holding segment.

FIG. 5 is a simplified diagram showing the flow of a fluid through an apparatus as described herein when used in a first permutation.

FIG. 6 is a simplified diagram showing the flow of a fluid through an apparatus as described herein when used in a second permutation.

FIG. 7 is a simplified diagram showing the flow of a fluid through an apparatus as described herein when used in a third permutation.

FIG. 8 is a simplified diagram showing the flow of a fluid through an apparatus as described herein when used in a fourth permutation.

FIG. 9 is a schematic showing a positional control system that may be used with the apparatus described herein.

### DETAILED DESCRIPTION

Reference will now be made in detail to various embodiments of the disclosed subject matter, one or more examples of which are set forth below. Each embodiment is provided by way of explanation of the subject matter, not limitation thereof. In fact, it will be apparent to those skilled in the art that various modifications and variations may be made in the present disclosure without departing from the scope or spirit of the subject matter. For instance, features illustrated or described as part of one embodiment may be used in another embodiment to yield a still further embodiment.

As used herein, the terms “analyte” and “sample” may be used interchangeably.

An analyte may be part of a fluid. Said fluid may comprise a biological fluid. A biological fluid may comprise whole cells and/or live cells and/or cell debris. The biological fluid may contain (or be derived from) a “bodily fluid”. The present disclosure encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples may include, but are not limited to, cell cultures, bodily fluids, and cell cultures from bodily fluids. Bodily fluids may be obtained from an organism, for example by puncture, or other collecting or sampling procedures.

In general, the present disclosure is directed to an apparatus which may aid in the analysis of an analyte and methods for using said apparatus. In some embodiments of the present disclosure, the apparatus may comprise five tubing segments and two fluidic switches connected thereby. The apparatus may further be coupled to instrumentation for analysis and/or stimulation. The method may comprise setting the apparatus to a first permutation of each of the first and second fluidic switches and introducing a fluid to the inlet segment of the apparatus, wherein the fluid comprises the analyte. Further, the method may comprise alternating between plurality of permutations, such as the first, second, third and fourth permutation to cycle fluid within the apparatus between the tubing segments.

The ability to repeatedly analyze the same analyte allows for significant benefits. For instance, within the field of cell biology, being able to observe a cell before and after stimulation, such as the introduction of a chemical agent, allows for scientists to examine the effects of the chemical agent, on not just a population scale, but also an individual scale. The ability to perform replicate measurements on an individual cell over a time scale of seconds to minutes can be used to improve resolution for taxonomic classification or study short time scale changes, such as photoadaptation. As described within the Background, sheath flow cytometers are often used for cell analysis. Sheath flow cytometers require the addition of a sheath fluid with laminar flow in order to form a uniform column of cells within the fluid. A downside of sheath flow cytometers is that they require low fluid throughput in order to maintain laminar flow of the sheath fluid. The presently described apparatus has a throughput within the analysis segment of 5 to 10 times that of traditional sheath flow cytometers.

That said, the presently described apparatus and methods of operation thereof are not particularly limited to applications within the field of cell biology. Thus, the present method enables a wide range of research techniques and is not limited in usefulness to one particular field.

In some embodiments of the present disclosure, the apparatus may comprise an inlet segment, an analysis segment, a holding segment, a bypass segment and an outlet segment. The apparatus may further comprise a plurality of fluidic switches. In some embodiments, the apparatus may comprise a first fluidic switch and a second fluidic switch. In such embodiments, the first fluidic switch may be defined by being operatively coupled with the inlet segment, and the second fluidic switch may be defined by being operatively coupled with the outlet segment.

Throughout the present disclosure, the term fluidic switch is used to denote a structure which may control the passage of fluid. Such valves may include pinch valves, switch valves, rotary valves or multiway selector valves. In some embodiments, the valve may be a multi-way valve, allowing for fluid flow to be diverted between multiple paths.

The inlet segment, analysis segment, holding segment, bypass segment and outlet segment may each comprise tubing with an interior lumen defined therein. The tubing may have an interior diameter between 0.3 and 1.5 mm, such as between 0.6 and 1.0 mm. The material of the tubing may include, but is not limited to, silicone, polytetrafluoroethylene, rubber or nylon. Further, each segment may, in embodiments, comprise a fluidic device, such as a microfluidic chip.

The analysis segment, holding segment and bypass segment may each have a first end and a second end, wherein the first end is defined as the end of the segment connected to the first fluidic switch, and the second end is defined as the end of the segment connected to the second fluidic switch.

Likewise, the inlet segment and the outlet segment may each have a first end. The first end of the inlet segment may be connected to the first fluidic switch, and the first end of the outlet segment may be connected to the second fluidic switch.

In some embodiments of the present disclosure, the analysis segment may be in proximity to analytical instrumentation. As used herein, analytical instrumentation is in proximity to the analysis segment when the analytical instrumentation is able to satisfactorily examine an analyte within the analysis segment. As an example, in embodiments wherein the analyte comprises whole cells, the analytical instrumentation may comprise a microscope. Thus, in such embodiments, the microscope is proximal to the analysis segment when a cell within the analysis segment can be examined by the microscope. Alternatively, in some aspects of the present invention, the analytical instrumentation may be integrated into the apparatus. For example, the fluid may flow through the analytical instrumentation. Said alternatively, the analytical instrumentation may be operatively coupled with the apparatus.

In some embodiments of the present invention, the apparatus is intended for use with fluid flowing therethrough, which may comprise a non-bodily fluid which contains biological material. For instance, the fluid may comprise a fluid which is not derived from bodily fluid and contains biological material including, but not limited to, whole cells, partial cells or cell contents, such as proteins, vesicles, lipids or carbohydrates or mixtures thereof.

In some embodiments of the present disclosure, the analyte may comprise a biological material. As a non-limiting example, the analyte may comprise whole cells, fragments of cells, exosomes, lipids, proteins, carbohydrates or organelles or mixtures thereof.

In some embodiments of the present disclosure, the analyte may comprise non-biological material. For instance, such material may include, but is not limited to, particulate, polymers, metals, alloys or lipids or mixtures thereof contained in a fluid carrier.

In some embodiments of the present disclosure, the analysis segment may comprise a capillary. Said capillary may be comprised of a low opacity glass, polymer or crystal. Additionally, the capillary may have different dimensions than the tubing segments of the other segments of the apparatus. For instance, while the tubing of the other segments of the apparatus (e.g., the inlet segment, the holding segment, the bypass segment, the outlet segment) may have a substantially isometric cross section, the capillary may have an anisometric cross section. The capillary may have an aspect ratio of length to width of greater than 5 to 1, such as 8 to 1, such as 10 to 1.

In embodiments, the analysis segment may comprise a microfluidic device, such as a microfluidic chip as previously described. While the specifics of the microfluidic chip are not particularly limited, the chip may comprise a plurality of channels, mixers or weirs. For instance, the chip may comprise at least two fluid pathways, wherein the two fluid pathways may be simultaneously visualized by analytical instrumentation as will be further described.

While the analytical instrumentation may comprise a microscope, the present disclosure is not so limited. For instance, the analytical instrumentation may comprise, among other things, a spectrophotometer, such as emission spectrophotometer or an excitation spectrophotometer, or a temperature probe. In general, one of skill in the art may appreciate that the analytical instrumentation may be adapted based upon the sample for analysis.

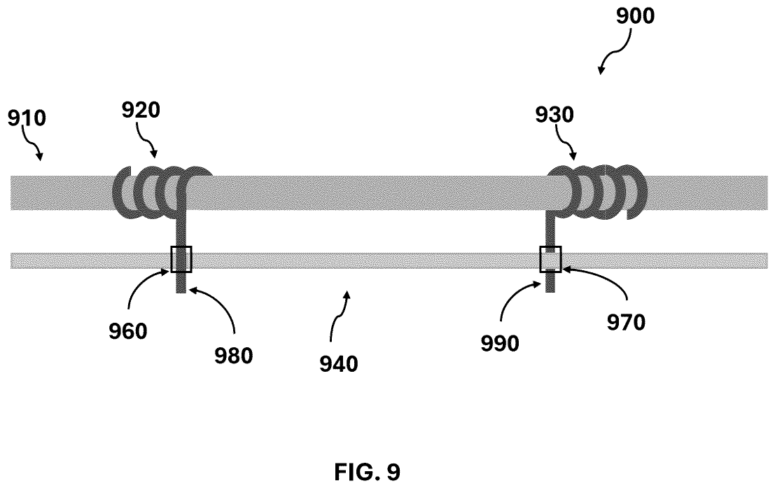

In some embodiments, the apparatus may comprise a positional control system which allows for positional adjustment of a sample within the analysis segment. Turning to FIG. 9, said positional control system 900 may comprise a control arm 910. Said control arm may be parallel with the analysis segment 940, and comprise two pressure arms 920, 930 each disposed on a different side of the control arm 910 and analysis segment 940. Rotation of the control arm 910 along its major axis may cause either of the first pressure arm 880 or second pressure arm 930 to contact a respective first pressure point 960 and second pressure point 970 of the analysis segment 940. The second pressure 970 point is distal to the first pressure point 960 relative to a first end of the analysis segment 940. Should pressure be applied to the first pressure point 960 of the analysis segment 940, fluid within the analysis segment 940 may move away from the first pressure point 960 due to compression of the analysis segment 940 at the first pressure point 960. Alternatively, pressure may be applied to the second pressure point 970 by the second pressure arm 930, which may cause a fluid within the analysis segment 940 to move away from the second pressure point 970 due to compression of the analysis segment 940 at the second pressure point 970. Thus, the position of an analyte within the analysis segment 940 may be adjusted as a result of application of pressure to either of the first pressure point 960 or second pressure point 970. In this manner, the position of an analyte may be finely adjusted, e.g., to position the analyte appropriately for analysis by an analytical instrumentation. The presently described positional control system is able to adjust the position of a sample within the analysis segment by 20 mm in either direction, with precision on the order of 10 microns.

Alternatively, positional control systems of the present disclosure may comprise plates which may compress the first and/or second pressure points 960, 970 of the analysis segment when activated by a servo motor. Further, a positional control system may comprise an acousto-fluidic device that can apply pressure with sound waves. Generally, the positional control system of the present disclosure comprises a means for applying pressure to a first pressure point and a second pressure point.

In some embodiments, the holding segment may comprise a structure defined by an interior lumen as described above. In some embodiments of the present disclosure, the holding segment may consist solely of a tube or pipe, e.g., with no additional components. In some embodiments of the present disclosure, the holding segment may allow for the stimulation of a sample by physical, chemical, thermodynamic or electromagnetic means. Generally, the holding segment may comprise a stimulator which applies a stimulus to fluid within the holding segment. As a non-limiting example, the holding segment may be subject to increased or decreased temperatures. To achieve increased or decreased temperatures, the apparatus of the present disclosure may include one or more thermally controllable devices in proximity to the holding segment. In another example, the holding segment may comprise a means for bombarding the sample with electromagnetic radiation, such as ultraviolet radiation. In this regard, the holding segment may be formed from a material that may receive the electromagnetic radiation such that the radiation stimulates the analyte.

While specific types of stimuli have been named above, it will be apparent to one of skill in the art that the apparatus, e.g., the holding segment, may comprise a wide variety of instruments useful for applying some stimulus to a sample, depending on the sample composition.

Further, the holding segment may comprise a device, such as a microfluidic chip as described above with respect to the analysis segment.

In embodiments wherein the apparatus comprises a plurality of fluidic switches, the apparatus may have a multitude of fluid flow permutations. In embodiments wherein the apparatus comprises two fluidic switches, the apparatus may have, e.g., four fluid flow permutations. A specific permutation may be obtained by altering the position of the first and second fluidic switches.

For instance, the first fluidic switch may be in a first position, wherein fluidic communication is established from the first end of the inlet segment to the first end of the analysis segment and from the first end of the holding segment to the first end of the bypass segment.

In a second position of the first fluidic switch, fluidic communication may be established between the first end of the inlet segment to the first end of the bypass segment and from the first end of the holding segment to the first end of the analysis segment.

Similarly, the second fluidic switch may be in either of a first position or a second position. In the first position, fluidic communication may be established from the second end of the analysis segment to the first end of the outlet segment, and from the second end of the bypass segment to the second end of the holding segment.

In a second position of the second fluidic switch, fluidic communication may be established from the second end of the analysis segment to the second end of the holding segment and from the second end of the bypass segment to the first end of the outlet segment.

For example, the first permutation may be one wherein the first fluidic switch is in a first position and the second fluidic switch is in a first position. In such a permutation, fluidic communication is established from the first end of the inlet segment to the first end of the analysis segment, from the first end of the holding segment to the first end of the bypass segment, from the second end of the analysis segment to the first end of the outlet segment, and from the second end of the bypass segment to the second end of the holding segment.

The second permutation may be defined by the first fluidic switch being in a second position and the second fluidic switch also being in a second position. In such a permutation, fluidic communication may be established from the first end of the inlet segment to the first end of the bypass segment, from the first end of the holding segment to the first end of the analysis segment, from the second end of the analysis segment to the second end of the holding segment, and from the second end of the bypass segment to the first end of the outlet segment.

The third permutation may be one where the first fluidic switch is in the first position, and the second fluidic switch is in the second position. In such a permutation, fluidic communication is established from the first end of the inlet segment to the first end of the analysis segment, from the first end of the holding segment to the first end of the bypass segment, from the second end of the analysis segment to the second end of the holding segment, and from the second end of the bypass segment to the first end of the outlet segment.

The fourth permutation may be one where the first fluidic switch being in the second position, and the second fluidic switch being in the first position. In such a permutation, fluidic communication is established from the first end of the inlet segment to the first end of the bypass segment, from the first end of the holding segment to the first end of the analysis segment, from the second end of the analysis segment to the first end of the outlet segment, and from the second end of the bypass segment to the second end of the holding segment.

One of skill in the art will be able to appreciate that the presently described number of permutations may be increased by a variety of means, such as increasing the number of fluidic switches and/or increasing the number of segments connected to the plurality of fluidic switches. For instance, while the present application describes an apparatus comprising two fluidic switches and four resulting permutations, it will become apparent to one of skill in the art that the present disclosure allows for the conception of apparatuses with an increased number of fluidic switches and fluid segments.

As a non-limiting example of a method of using the presently described apparatus, a user may first introduce a fluid containing an analyte of interest to the apparatus. The flow rate of the fluid is not particularly limited, though it may depend on the pressure of the fluid being introduced to the apparatus, as well as the specific construction of the apparatus, i.e., the internal diameter of the tubing within the apparatus. The flow rate may be from 50 microliters per minute to 20 milliliters per minute, such as from 95 microliters per minute to 1 milliliter per minute. For detailed analysis of the analyte, slower flow rates may be used, such as from 50 microliters per minute to 800 microliters per minute, such as from 100 microliters per minute to 500 microliters per minute. In situations where the user wishes to operate the apparatus in a flow cytometer mode, flow rates may be from 1 to 20 milliliters per minute, such as from 5 to 10 milliliters per minute.

If the apparatus is in the first permutation, the analyte will flow into the analysis segment, i.e., through the first fluidic switch in the first position. At this point, the user may decide to continue analysis of that specific analyte or continue to view new analytes. Should the user wish to view new analytes, the apparatus may be left in the first permutation. However, the user may instead place the apparatus into the second permutation. In the second permutation, fluid flow from the inlet segment is disconnected from the analysis segment and is instead routed to the outlet segment via the bypass segment. The analyte may remain within the fluid within the analysis segment. Should the position of the analyte be unsatisfactory, means for adjusting the position of the analyte may be used, as is described above. Next, if the user wishes to subject the analyte to some stimulus, the apparatus may be placed into the third permutation, wherein fluid in the analysis segment is directed through the second fluidic switch to the holding segment. The fluid flow rate may be increased in this step to quickly pass the sample through the apparatus. For instance, the flow rate may be increased to between 1 and 20 milliliters per minute, such as between 5 and 10 milliliters per minute. As stated above, the holding segment may comprise solely tubes, or an instrument for effecting some stimulation to the analyte. After holding or stimulation, the apparatus may be placed into the fourth permutation, wherein fluid is directed from the holding segment through the first fluidic switch, back to the analysis segment. At this point, the user may once again analyze the same cell after it has been held for some duration of time or perturbed in some manner.

Details on the apparatus and method of operation as described herein may be exemplified by the figures, and accompanying description that follows. The figures and the description which follow serve as an example of the apparatus and method of operation, not as a limitation to any particular feature of the present disclosure.

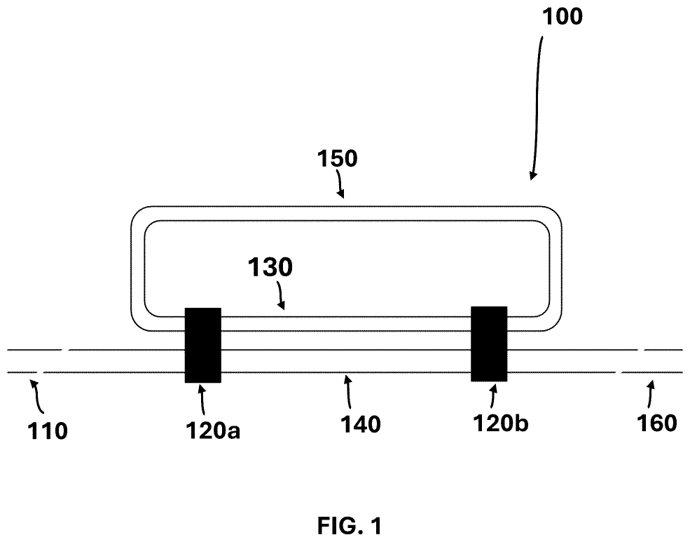

FIG. 1 depicts a simplified diagram of the apparatus 100 as described herein. Said apparatus 100 comprises an inlet segment 110 which is operatively coupled with first fluidic switch 120a. First fluidic switch 120a dictates whether fluid within the apparatus is directed to bypass segment 130, or analysis segment 140. In the first case that fluid is directed to bypass segment 130, fluid flows within bypass segment 130 to second fluidic switch 120b. At second fluidic switch 120b, fluid may be directed to either the holding segment 150, or the outlet segment 160. In the first case where fluid is directed by fluidic switch 120b to holding segment 150, fluid will return to fluidic switch 120a.

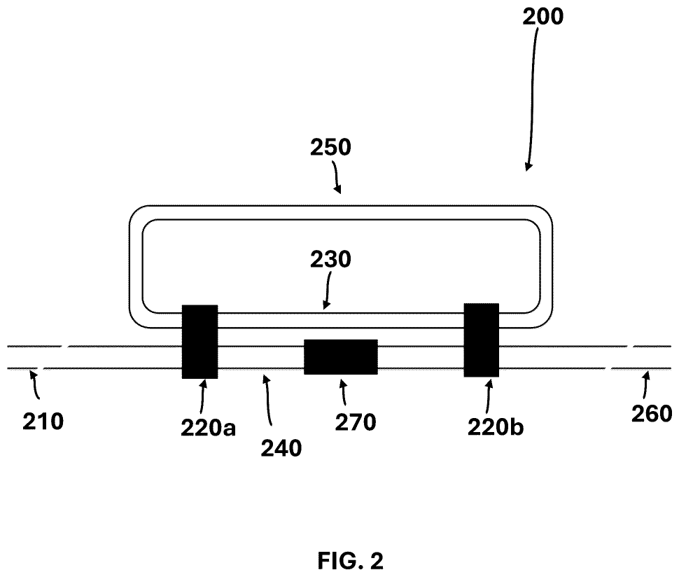

FIG. 2 has a similar structure as the apparatus shown in FIG. 1. However, if fluid at first fluidic switch 220a is directed to the analysis segment 240, fluid will flow into analysis instrument 240. As described above, analysis instrument 240 may comprise a variety of components for use in a variety of applications. After leaving, e.g., flowing past, analysis instrument 240, fluid is directed to second fluidic switch 220b. At second fluidic switch 220b, fluid may be directed by second fluidic switch 220b to either outlet segment 260 or holding segment 250.

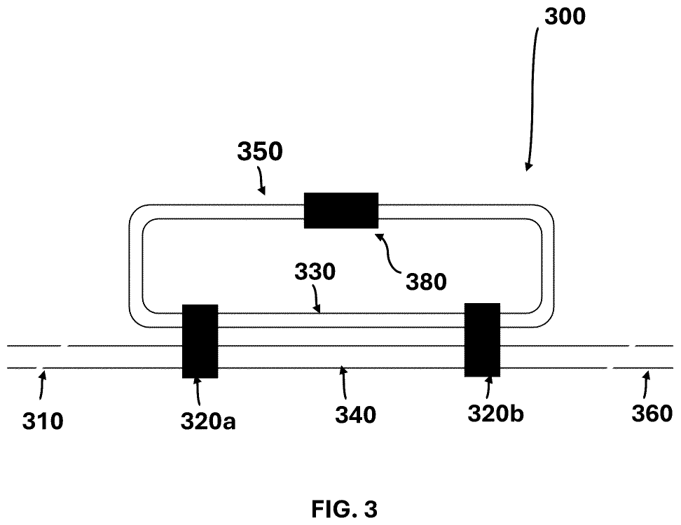

FIG. 3 has a similar structure as the apparatus shown in FIG. 1. However, if fluid is directed to holding segment 350 after passing through second fluidic switch 320b, fluid will encounter a perturbation instrument 380. As described above, the perturbation instrument 380, also referred to herein as a stimulator, may comprise a variety of instruments useful for performing manipulations to a sample contained within the fluid. After passing through perturbation instrument 380, fluid is directed back to first fluidic switch 320a.

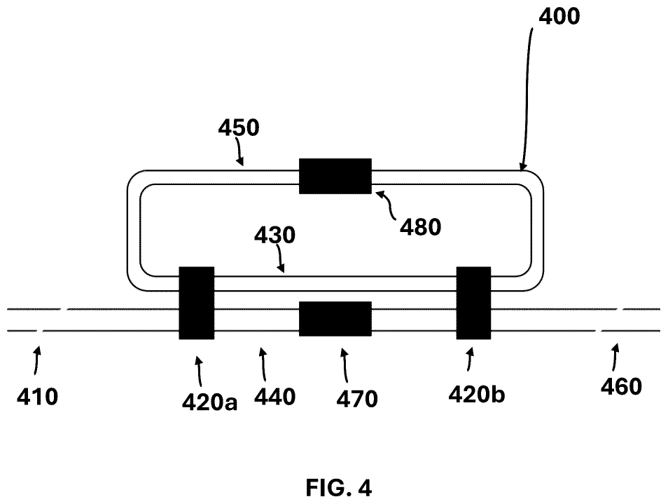

The diagram shown in FIG. 4 depicts an exemplary apparatus similar in structure to the apparatus shown in FIG. 1. However, fluid may encounter both an analysis instrument 470 and a perturbation instrument 480.

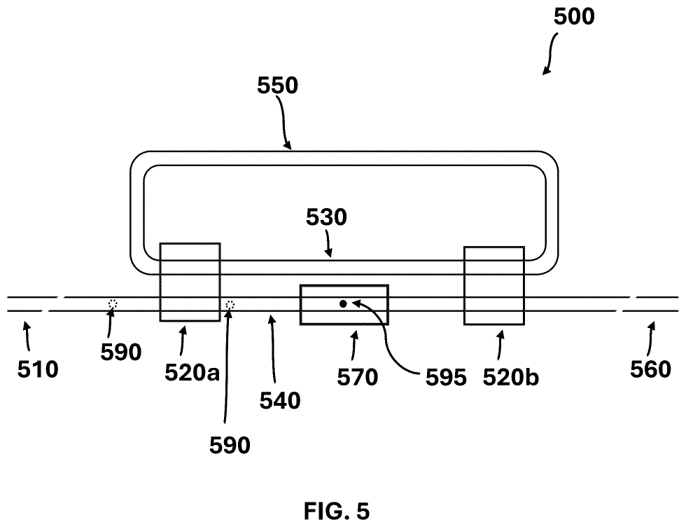

FIGS. 5-8 are directed to embodiments as described herein where the apparatus comprises at least two fluidic switches. Further, FIGS. 5-8 demonstrate how a series of permutations with respect to the close/open states of the fluidic switches affect fluid flow.

FIG. 5 is a diagram of apparatus 500. In the permutation of apparatus 500 shown, both fluidic switches are in the first position. First, fluid flows into inlet segment 510. Fluid then passes through first fluidic switch 520a into analysis segment 540. The flow of the fluid is shown by previous analyte positions 590, and current analyte position 595. When the current analyte position 595 is inside analysis instrument 570, a user of apparatus 500 may allow fluid to pass second fluidic switch 520b, which is in the first position. The fluid may then flow to outlet segment 560. Alternatively, the user may switch the apparatus 500 to the second permutation wherein fluidic switches 520a and 520b are in the second position.

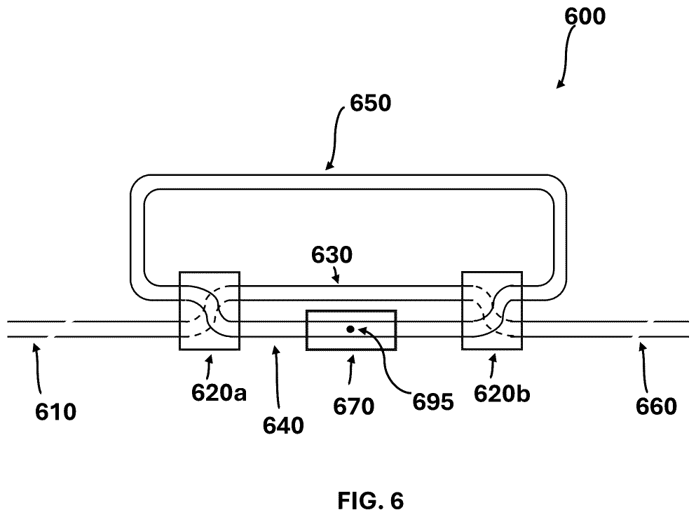

FIG. 6 is a diagram of apparatus 600. Apparatus 600 as shown is in the second permutation, wherein both fluidic switches 620a and 620b are in the second position. In this instance, fluid does not move, i.e., there is no fluid flow, within the analysis segment 640 or the analysis instrument 670, allowing the user to further analyze the fluid in current analyte position 695. Meanwhile, fluid from inlet segment 610 is directed through first fluidic switch 620a to bypass segment 630, through second fluidic switch 620b and to outlet segment 660. Should the user wish to view fluid from inlet segment 610 while saving a portion of the fluid in current analyte position 695, the user may switch apparatus 600 to the third permutation.

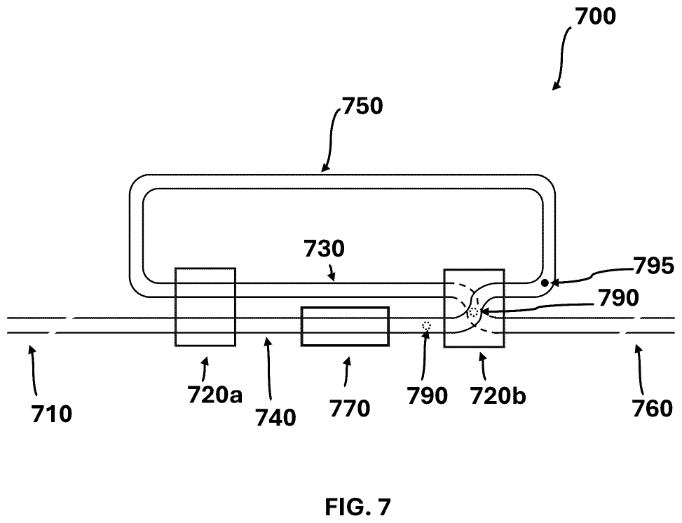

FIG. 7 is a diagram of apparatus 700. The apparatus 700 as shown is in the third permutation wherein the first fluidic switch 720a is in the first position, and the second fluidic switch 720b is in the second position. As shown by the previous analyte positions 790 and the current analyte position 795, fluid is directed from the inlet segment 710 to the first fluidic switch 720a to the analysis segment 740, through the analysis instrument 770 and the second fluidic switch 720b into the holding segment 750. Further, fluid from holding segment 750 is directed to bypass segment 730 through first fluidic switch 720a, through second fluidic switch 720b to outlet segment 760. However, should the user wish to analyze a portion of the fluid within holding segment 750, the user may switch the apparatus to the fourth permutation wherein the first fluidic switch 720a is in the second position, and the second fluidic switch 720b is in the first position.

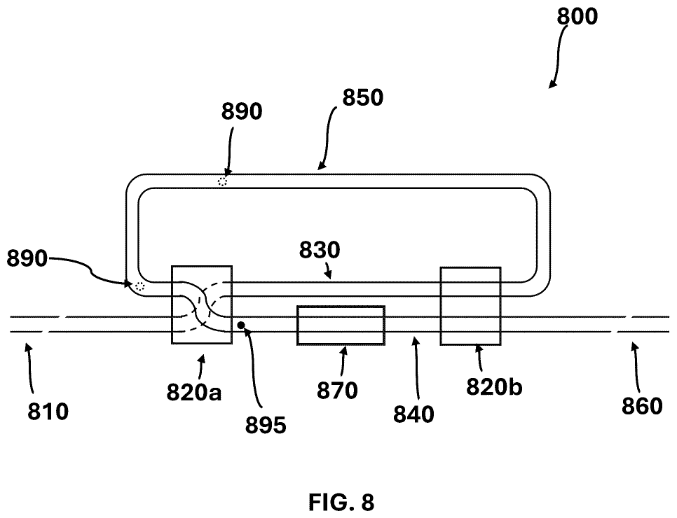

FIG. 8 is a diagram of apparatus 800. The apparatus 800 as shown is in the fourth permutation wherein the first fluidic switch 820a is in the second position, and the second fluidic switch 820b is in the first position. In this permutation, fluid flows from the holding segment 850 to the first fluidic switch 820a to the analysis segment 840. Fluid may then pass to the analysis instrument 870 for repeated analysis.

FIG. 9 is a schematic of an example of a control system 900 which can be used to apply pressure to the first or second pressure points. Control arm 910 runs substantially parallel to analysis segment 940. Control arm 910 comprises pressure arms 920, 930 disposed on control arm 910. In embodiments, the pressure arms 920, 930 may comprise coils wrapped around the control arm 910. Pressure arms 920 and 930 are wrapped around control arm 910 in an antiparallel manner. Pressure arms 920 and 930 each terminate at one end in pressure points 960 and 970 respectively. Pressure arms 920 and 930 may be used to apply pressure to opposing sides of analysis segment 940 at pressure points 960 and 970. For instance, rotating control arm 910 clockwise may cause pressure arm 930 to apply pressure to pressure point 970, thereby pushing fluid within analysis segment 940 toward pressure point 960. Alternatively, if control arm 910 is rotated anticlockwise, pressure arm 920 may apply pressure to pressure point 960, causing fluid within analysis segment 940 to flow toward pressure point 970.

The present invention may be better understood with reference to the examples set forth below.

### Example 1

While certain embodiments of the disclosed subject matter have been described using specific terms, such description is for illustrative purposes only, and it is to be understood that changes and variations may be made without departing from the spirit or scope of the subject matter.

Phytoplankton are microscopic unicellular organisms, and they are ubiquitous in marine and freshwater environments across the globe. They play a critical role in primary production, carbon fixation, and O₂ production through photosynthesis. Phytoplankton comprise an abundant variety of genera and species, and the taxonomic makeup of a community can be an important indicator of environmental health. Therefore, it is important to be able to study phytoplankton communities in the lab and in the field. Phytoplankton are very diverse; species vary in size from <1 μm to >100 μm, and different species contain different accessory pigments, which results in each species having a unique fluorescence spectrum, so phytoplankton are commonly classified by size and pigmentation. However, cells will change in size throughout their lifetime as they grow and divide, and they can also alter their pigmentation in response to environmental factors, so it is important to understand the variability in size and pigmentation between individuals of the same species, as well as the variability for a single individual at different points in time. Automated methods of single cell and bulk analysis, including flow-through techniques like flow cytometry are popular tools for studying these changes. A limitation of traditional flow-through analysis is that each sample can only be observed once, and it is impossible to track individual cells following analysis.

In order to repeatedly analyze the same phytoplankton, the apparatus as described herein may be utilized. For instance, phytoplankton may be contained within a fluid. Said fluid may enter the apparatus at the inlet segment 510. The phytoplankton may then pass through the first fluidic switch 520a to the analysis segment 540. At the analysis segment 540, a microscope or spectrophotometer may be used to observe some property of the phytoplankton. Should the user wish to view the phytoplankton for longer, the apparatus may be switched into the second permutation. Thereafter, the user may switch the apparatus to the third permutation. In the third permutation, the user may subject the phytoplankton to some treatment, such as irradiation. After the treatment, the user may then switch the apparatus to the fourth permutation.

Additionally, the presently utilized system allows for the repeated analysis of samples without changing flow directions. That is, the fluid direction may remain the same through the inlet segment and the outlet segment.

In further embodiments, the present invention allows for micro-adjustments of sample positioning within the analysis segment. If, for instance, the phytoplankton is slightly misaligned with the lens of the microscope, the user may use the positional control system described above in order to adjust the positioning of the phytoplankton.

### Example 2

The capillary was constructed from square cross-section glass capillary tubing, having an inner dimension width (ID) of 300 μm and an outer dimension width (OD) of 600 μm, cut to a length of 5 cm (8330 VitroCom, Mountain Lakes, NJ). The ends of the capillary were fit into round silicone tubing with a 1/32″ ID and a 3/32″ OD (TBGM100, NResearch Inc., West Caldwell, NJ). To provide additional compression and prevent leaks, small bands were cut from the TBGM100 tubing (about 5 mm wide) and stretched over the ends of the silicone tubing where it overlapped the capillary. Solution comprising a sample was pumped through the flow cell using a syringe pump (NE300, New Era Pump Systems Inc., Farmingdale, NY) and 20 mL sterile syringes (HSW Norm-Ject, VWR, Radnor, PA). The typical flow rate was 100 μL/min, corresponding to a speed of approximately 18.5 mm/s, but the system is capable of operating at flow rates up to 10 mL/min.

The tubing was divided into five sections, connected with barbed Y-connectors: an inlet segment 510 from the syringe pump, the analysis segment 140 which is the section containing the capillary, a bypass segment 130, a holding segment 150, and an outlet segment 160. Flow through the system was controlled using first and second fluidic switches 120a, 120b comprising 4-tube solenoid pinch valves (225P071-11, NResearch, West Caldwell, NJ), with each valve having 2 tubes open at a time. With two such valves, there are four possible valve permutations. The tubing and valves were connected such that with both valves open, fluid flowed straight through the capillary and out to waste. With both valves closed, the analysis segment 140 comprising the capillary was isolated, and flow was diverted through the bypass segment 130 to waste. With the first fluidic switch 120a open and the second fluidic switch 120b closed, samples could be loaded into the holding segment 150 after passing through the capillary. Finally, with the first fluidic switch 120a closed and the second fluidic switch 120b open, samples in the holding segment 150 could be brought back through the capillary without reversing the direction of flow by diverting flow through the bypass segment 130 into the holding segment 150. The bypass section included an inline 0.22 μm filter to remove any particulate which ensured that nothing was inadvertently loaded into the holding segment 150 from the bypass segment 130 during this process.

As discussed above, the apparatus has several advantages over traditional sheath flow cytometers. For instance, the apparatus was much less likely to get clogged, and a much larger range of cell sizes could be analyzed without needing to modify hardware such as nozzle tips.

To reposition samples within the capillary while the valves remained in the closed position, torsion springs (9271K579, McMaster-Carr, Elmhurst, IL) were mounted on a ¼″ shaft and placed in contact with the tubing on either side of the capillary; the shaft was coupled to a servo motor (HS-625 MG, Hitec RCD, San Diego, CA) that could rotate 90° in either direction. The springs were positioned so that a CW rotation compressed the tubing on the “upstream” side of the capillary, pushing fluid forward, and a CCW rotation compressed the tubing on the opposite side, pushing fluid in reverse. The ends of the springs were fitted with rubber tips at the contact points with the silicone tubing to prevent wear and to increase the contact surface area. The servo provides fine control over the position of beads within the analysis segment 140, with a range of motion of up to 20 mm and precision on the order of 10 μm. This makes it possible to reposition a single bead within a laser beam and hold it there.

The entire system was controlled using a versatile, commercially available system-on-a-chip hardware platform, the Red Pitaya STEMlab 125-14 ($427, Red Pitaya, Solkan, Slovenia). This device incorporates two high speed analog inputs (125 MSps, 14-bit resolution), multiple digital GPIO pins, an ethernet connection for remote connections and high-speed data transfer, and a field programmable gate array (FPGA). The device runs on the Red Pitaya operating system, a customized version of Ubuntu Linux that includes a variety of pre-built software applications including a digital oscilloscope, logic analyzer, spectrum analyzer, and data streaming application.

Illumination was kept consistent throughout all parts of the capillary volume. The light source used was a 450 nm diode laser (11041069, LaserLands.net, Besram Technology, Wuhan, China) with a nominal power of 80 mW and elliptical beam approximately 1.8×3.0 mm. This laser was powered at 5.0 V and 90 mA by an independent benchtop power supply (GPR 11H30-D, GW Instek). An adjustable rectangular slit (Linos Photonics, Milford, MA) was employed to spatially filter the beam to the desired size (approximately 0.6×1.1 mm). This decision minimized the number of surfaces that could potentially contribute to the background of scattered light. Also, since the beam is collimated passing through the capillary, a more uniform illumination of the entire analyzed volume was obtained. After passing through the capillary, the beam was terminated on a custom 1.0 mm wide beam block 3D printed from black PETG. The beam block was placed immediately in front of the collection lens, a 1″ asphere (ACL2520U, Thorlabs, Newton, NJ) (f/0.8) with its focal point aligned to the center of the capillary. The collection lens has a NA of 0.60, with the beam block obscuring the central half-angle of ˜3°. Fluorescence and forward light scattering signals from samples passing through the beam were collected and collimated by the asphere. A 2″ dichroic beamsplitter (BS625LP, Thorlabs, Newton, NJ) was used to separate the signals. The transmitted light (red fluorescence) was filtered using a 665-735 nm bandpass filter (FF01-698/70-25, Semrock, Rochester, NY) then focused onto an APD detector (APD440A, Thorlabs, Newton, NJ) using a 1″ f/3 lens. The reflected light (blue scattering) was passed through a second, neutral beamsplitter (BS013, Thorlabs, Newton, NJ), with the transmitted beam passed through a 450 nm bandpass filter (Edmund Optics, Barrington, NJ) then focused onto an APD (APD210A-AC, Thorlabs, Newton, NJ) using an identical f/3 lens and the reflected beam focused onto a USB camera (ELP-USBFHD01M-MF40, Amazon.com) using a third identical lens. The system was aligned so that both detectors and the camera were parfocal with one another.

The fluorescence APD had an adjustable gain, which was always set to the maximum level; the output of this detector was connected directly to one of the high-speed inputs on the Red Pitaya. The scattering APD was AC coupled to remove the background signal from light scattered off the walls of the capillary, or other optical elements; the output of this detector was then passed through a benchtop amplifier (SR-560, Stanford Research Systems, Sunnyvale, CA) to remove high frequency noise with a 10 KHz low-pass filter and apply a 10× gain.

Polystyrene microspheres (Polysciences, Warrington, PA) were used as a standard to measure instrument performance: 10 μm and 25 μm Fluoresbrite™ Yellow-Green beads (YG10 and YG25), 6 μm Ruby Red beads (RR6) and 6 μm undyed beads (UD6). Samples were prepared by diluting a few drops of bead stock in 10 mL of filtered methanol to sterilize and dilute the beads. The resulting bead concentrate was then diluted to a final concentration of approximately 10 bead/mL (determined using the flow cytometer function of the apparatus). Since the polystyrene beads have a density of 1.05 g/cm3 (Polysciences, 2018), to ensure that they would be neutrally buoyant, bead samples were prepared using a 27% w/w mixture of glycerol in DI water (Volk and Kähler, 2018). This mixture was then filtered through a sterile syringe filter (Fisherbrand 09-720-511, Thermo Fisher Scientific, Waltham, MA) to remove any particulate larger than 0.22 μm. Hemiselmis aquamarina (RCC 4102) was obtained from the Roscoff Culture Collection (RCC).

Thalassiosira weissflogii and Proteomonas sulcata (CCMP1175) were obtained from the National Center for Marine Algae and Microbiota (NCMA) at the Bigelow Laboratory for Ocean Sciences. Phytoplankton were grown in the appropriate media made from metal and nutrient kits purchased from the NCMA. H. aquamarina was grown in K media, T. weissflogii was grown in a mixture of L2+Si and F/2-Si media, and P. sulcata was grown in F/2-Si media. Stock cultures were grown in a reach-in incubator at 20° C. Light was supplied on 12:12 hour light-dark cycle at 80 μmol photon m-2 s-1 measured inside the bottle with a LI-250A light meter (LI-COR Biosciences Inc., Lincoln, NE USA).

For analysis in the apparatus, individual samples were prepared by diluting 20-50 μL of the stock in 20-50 mL of 0.22 μm filtered seawater. Samples were kept in the dark for at least 30 minutes prior to analysis.

The Red Pitaya supports custom programming with a variety of languages, including Python. In the experiment, the high-speed analog inputs were used to record data from the APD detectors, and the digital GPIO pins were used to control the valves and servo. Two custom programs were developed to control the apparatus using the Python API: one allowing for trapping, replicate analysis, and sample tracking; and one for simple flow cytometer operation. Each program included several user-defined parameters including sample flow rate, signal triggering threshold, trigger source (scatter or fluorescence channel), number of samples to record, and number of replicates per sample. In trapping mode, when a trigger was detected, the first and second fluidic switches 120a, 120b would be closed, trapping the bead within the analysis segment 140. A short delay (0.5-5 ms, calculated based on flow rate) was used to ensure that the bead had completely passed through the analysis segment 140, so that the data collected from the initial pass was not disturbed by trapping. Immediately after trapping, the motor was used to bring the bead back to the center of the analysis segment 140, until the trigger signal was observed again. The rotation required to reach this center position was recorded and left and right limits were set at fixed angles in either direction relative to this center position. Beginning at the left (upstream) limit, the motor was rotated at its maximum speed to the right (downstream) limit, sending the bead through the analyzer. Then the motor was held at the right limit while the data from both channels was recorded, then sent back to the left limit to send the bead through the analysis segment 140 in reverse. Again, the motor was held in place while data was recorded, then this process was repeated until the desired number of passes had been recorded. After exiting the passing routine, a file was saved with the center position and the scattering and fluorescence signals from each pass, including the initial trapping pass. Typical trappings consisted of 10 passes in each direction, lasting a total of approximately 10 seconds; during this time, all sample flow was directed to waste through the bypass, so low flow rates were preferred to avoid wasting sample. Sample “tracking” is implemented by initializing a ‘load position’ timer variable for each bead as it leaves the analysis segment 140. Since the volume of tubing in each segment is fixed, the location of a bead at a given point in time can be approximated based on the flow rate and valve state. When the first and second fluidic switches 120a, 120b are in the closed state, all position timers are paused, otherwise they are incremented and tracked within a loop until a bead that has been selected for holding reaches the intersection point at the second fluidic switch 120b, or a trapping event occurs. Once a bead passes this intersection, its ‘load position’ timer is deleted; beads that are loaded into the holding loop receive a new ‘loop position’ timer variable that only increments when the valves are in the “loop fill” or “loop empty” state; this ‘loop position’ variable is also used to track the fill state of the holding segment 150. As samples are removed from the holding segment 150 and moved back into the analysis segment 140, their ‘loop timer’ variables are deleted; they will be assigned new timer variables after they leave the analyzer, and once they re-enter the holding loop. With this apparatus it is possible to recirculate the same cell through the holding segment 150 and analysis segment 140 an arbitrary number of times. The apparatus was also able to operate in a simple flow cytometer mode in which the valves remained in the open position, and no trapping was performed. Instead, the user specified the flow rate, trigger level, trigger source and desired counting duration. Upon triggering, data from each detector was processed with a simple algorithm to identify the height and width of the signal on each channel. These four parameters were stored for each triggering event and saved to a file at the end of the specified duration. To avoid coincidences of multiple beads within the analyzer simultaneously, sample concentrations were limited to 100 ml⁻¹, so at the maximum flow rate of 10 mL/min, the maximum expected count rate is <20 s⁻¹. To ensure robustness, the program was capable of processing up to 500 triggering events per second.

What is claimed is:

1. An apparatus for repeat analysis of an analyte, the apparatus comprising:
an inlet segment comprising a first end;
an analysis segment comprising a first end and a second end;
an outlet segment comprising a first end;
a bypass segment comprising a first end and a second end;
a holding segment comprising a first end and a second end; and
a first fluidic switch and a second fluidic switch,
wherein the first fluidic switch establishes fluidic communication from the first end of the inlet segment to the first end of the analysis segment, and from the first end of the holding segment to the first end of the bypass segment,
or from the first end of the inlet segment to the first end of the bypass segment, and from the first end of the holding segment to the first end of the analysis segment,
and wherein the second fluidic switch establishes fluidic communication from the second end of the analysis segment to the first end of the outlet segment, and from the second end of the bypass segment to the second end of the holding segment,
or from the second end of the analysis segment to the second end of the holding segment, and from the second end of the bypass segment to the first end of the outlet segment.

2. The apparatus of claim 1, wherein the analysis segment comprises a capillary.

3. The apparatus of claim 1, wherein the holding segment comprises a stimulator.

4. The apparatus of claim 1, further comprising analytical instrumentation proximal to the analysis segment.

5. The apparatus of claim 1, further comprising a microscope proximal to the analysis segment.

6. The apparatus of claim 1, further comprising a spectrophotometer proximal to the analysis segment.

7. The apparatus of claim 1, wherein the first fluidic switch establishes fluidic communication from the first end of the inlet segment to the first end of the analysis segment, and from the first end of the holding segment to the first end of the bypass segment, and the second fluidic switch establishes communication from the second end of the analysis segment to the first end of the outlet segment, and from the second end of the bypass segment to the second end of the holding segment.

8. The apparatus of claim 1, wherein the first fluidic switch establishes fluidic communication from the first end of the inlet segment to the first end of the bypass segment, and from the first end of the holding segment to the first end of the analysis segment,
and wherein the second fluidic switch establishes fluidic communication from the second end of the analysis segment to the second end of the holding segment, and from the second end of the bypass segment to the first end of the outlet segment.

9. A method for analysis of an analyte, the method comprising:
setting the apparatus of claim 1 to a first permutation of each of the first and second fluidic switches; and
introducing a fluid to the inlet segment of the apparatus, wherein the fluid comprises the analyte.

10. The method of claim 9, wherein the fluid flows through the apparatus at a rate of from 100 and 500 microliters per minute.

11. The method of claim 9, wherein the fluid flows through the apparatus at a rate of from 1 and 20 milliliters per minute.

12. The method of claim 9, further comprising setting the apparatus to a second permutation of each of the first and second fluidic switches.

13. The method of claim 9, further comprising setting the apparatus to a third permutation of each of the first and second fluidic switches.

14. The method of claim 12, further comprising setting the apparatus to a third permutation of each of the first and second fluidic switches.

15. The method of claim 14, further comprising setting the apparatus to a fourth permutation of each of the first and second fluidic switches.

16. The method of claim 13, further comprising setting the apparatus to a fourth permutation of each of the first and second fluidic switches.

17. The method of claim 14, wherein the holding segment comprises a stimulator.

18. The method of claim 9, wherein the analyte comprises a cell.

19. The method of claim 16, comprising setting the apparatus to a second permutation.
